(19) United States
(12) Patent Application Publication
(10) Pub. No.: US 2022/0160798 A1
(43) Pub. Date: May 26, 2022

(54) METHOD FOR PRODUCING SPIRULINA EXTRACT, AND SPIRULINA EXTRACT-CONTAINING PHARMACEUTICAL COMPOSITION AND HEALTH FUNCTIONAL FOOD FOR IMPROVING COGNITIVE ABILITY

(71) Applicant: KOREA INSTITUTE OF OCEAN & SCIENCE TECHNOLOGY, Busan (KR)

(72) Inventors: Do-Hyung KANG, Jeju-do (KR); Taeko KIM, Jeju-do (KR); Youngdeuk LEE, Jeju-do (KR); Woon-Yong CHOI, Jeju-do (KR); Yong Kyun YOU, Seoul (KR); Won Kyu LEE, Seoul (KR); Areumi PARK, Busan (KR)

(21) Appl. No.: 17/436,356

(22) PCT Filed: Feb. 19, 2020
(86) PCT No.: PCT/KR2020/002354

§ 371 Date: Sep. 3, 2021

(30) Foreign Application Priority Data
Mar. 5, 2019 (KR) 10-2019-0025275

(51) Int. Cl. A61K 35/748; A23L 33/135; B01D 11/02; A61K 9/19

(52) U.S. Cl. CPC A61K 35/748; A23L 33/135; A61K 9/19; B01D 11/0288; B01D 11/028

(57) ABSTRACT

An object of the present invention is to preserve the total content of chlorophyll in an extract while increasing the extraction yield of a Spirulina extract. Another object of the present invention is to provide a pharmaceutical composition and health functional food for prevention or treatment of degenerative cranial nerve diseases containing a Spirulina extract as an active ingredient, and to provide a method for treating degenerative cranial nerve diseases using a Spirulina extract. The present invention provides a method for producing a Spirulina extract including the steps of (a) preparing a Spirulina powder; (b) adding 50% to 80% of ethanol to the Spirulina powder and performing an ultrasonic pretreatment at 15° C. to 35° C.; (c) extracting a Spirulina extract at 50° C. to 80° C.; (d) vacuum-concentrating the Spirulina extract; and (e) freeze-drying the vacuum-concentrated Spirulina extract.

### TECHNICAL FIELD

The present invention relates to a method for producing a Spirulina extract, a pharmaceutical composition and health functional food for preventing or treating degenerative cranial nerve diseases containing a Spirulina extract, and a method for treating degenerative cranial nerve diseases using a Spirulina extract. More specifically, the present invention relates to a method for producing a Spirulina extract, a pharmaceutical composition and health functional food for preventing or treating degenerative cranial nerve diseases containing a Spirulina extract, and a method for treating degenerative cranial nerve diseases using a Spirulina extract capable of preserving a total content of chlorophyll contained in the Spirulina extract while enhancing an extraction yield.

### BACKGROUND ART

Dementia is a disease which is characterized by overall disability in mental functions, such as memory disorders, judgment loss and deserts the human life. The prevalence of dementia is about 8% from the elderly of 65 years old or more in Korea, and it is estimated that about 0.35 million of about 4.3 million of the elderly population in Korea suffer from dementia diseases (Seoul Dementia Center, 2015 Dementia Patient Survey Report).

As the rapid aging of the population progressed, the number of dementia patients is expected to surge, and as a result, the measures according to dementia are urgent. In addition, while the elderly medical expenses continued to grow, in 2010, the elderly dementia expenses have surged by 32% compared to the previous year (Health Insurance Review & Assessment Service, 2010 Medical expenses statistics).

Alzheimer's disease (AD), which is the biggest part as 71% of dementia types, was not exactly found that the cause of the onset, but when the brain tissue in Alzheimer's disease patients is observed, it can be seen that the damage of the cholinergic nerve seriously occurs. As such, the theory to describe the cause of Alzheimer's disease as the damage to the cholinergic nerve is called Colin hypothesis, and recently, in order to induce a functional degradation of acetylcholine, a lot of attempts to inhibit the activity of acetylcholinesterase have been made. In addition, in the brain of the patient with Alzheimer's disease, due to the activity of □ and □-secretase, an abnormal toxic protein, which is amyloid beta, is deposited to form neuritic plaques and neurofibrillary tangles, thereby causing damage to cognitive ability.

Currently, drugs used as a therapeutic agent of Alzheimer's disease include tacrine, donepezil, rivastigmine, and galantamine as acetylcholinesterase inhibitors. Among them, galantamine has most recently been used by the approval of Food and Drug Administration (FDA) in US, and inhibits the decomposition of acetylcholine to maintain the acetylcholine concentration in synapses, thereby improving the cognitive ability.

However, these medicines are a single preparation drug made through synthesis, and since the effect is temporary and weak and these medicines do not selectively act only on the brain, there is a disadvantage that it is difficult to be administered for a long time due to various side effects. In addition, galantamine is suspected of safety due to toxicity and side effects, and these side effects deteriorate consumers' reliability. Therefore, there is a need for developing drugs made of natural functional materials with more safety and excellent efficacy. In addition, an active inhibitory material of □-secretase associated with the most expected amyloid beta deposition as a next generation Alzheimer's therapeutic agent is not yet developed.

Meanwhile, Spirulina is a microorganism that belongs to very small algae vigorously growing wild on the surface of the tropical regional alkaline lake, such as Chad Lake of Africa and Texko Lake of Mexico. In Spirulina cells, since there is a large amount of chlorophyll, picochein, or the like, the solar rays are absorbed to actively perform carbon dioxide assimilation. The pigment such as chlorophyll, picochein, or the like has blue, and from old times, Spirulina was classified as blue-green algae.

Spirulina, as a microorganism which can be eat by the human, contains 55 to 70% of protein, 6 to 9% of fat, and 15 to 20% of carbohydrate, and contains large amounts of minerals, vitamins, fibrous and pigment components. Spirulina contains not only a high content of protein, but also contains eight essential amino acids, and among fat components, free-fatty acid reaches 70 to 80%, and fatty acid such as linoleic acid, □-linolenic acid or the like has a large portion. The carbohydrate content of Spirulina is small, but mainly consists of rhamnose and glycogen, and is absorbed without the help of insulin to be used as an energy source of diabetic patients. Local people have collected these microalgae and used the microalgae as edible food for a long time, and as a nutritional study, it has been found that a high content of proteins and various nutrient components including amino acids are constituted as very beneficial ingredients for human health.

Meanwhile, as a prior art related with the present invention, in Korean Patent Registration No. 10-1418545, there is provided a pharmaceutical composition for prevention and treatment of neurodegenerative brain diseases, but there is no composition for treating degenerative cranial nerve diseases using natural functional materials like the present invention.

### DISCLOSURE

### Technical Problem

An object of the present invention is to provide a method for producing a Spirulina extract capable of increasing the extraction yield of the Spirulina extract for prevention or treatment of degenerative cranial nerve diseases.

Another object of the present invention is to provide a method for producing a Spirulina extract capable of preserving the total content of chlorophyll included in the extract while increasing the extraction yield of the Spirulina extract.

Yet another object of the present invention is to provide a pharmaceutical composition and health functional food capable of preventing or treating degenerative cranial nerve diseases containing a Spirulina extract as an active ingredient.

### Technical Solution

To achieve the objects, the present invention provides a method for producing a Spirulina extract including the steps of (a) preparing a Spirulina powder; (b) adding 50% to 80% of ethanol to the Spirulina powder and performing an ultrasonic pretreatment at 15° C. to 35° C.; (c) extracting a Spirulina extract at 50° C. to 80° C.; (d) vacuum-concentrating the Spirulina extract; and (e) freeze-drying the vacuum-concentrated Spirulina extract.

The Spirulina in step (a) may be any one selected from the group consisting of Spirulina maxima, Spirulina platensis, Spirulina geitleri, Spirulina Siamese, Spirulina major, Spirulina subsalsa, Spirulina princes, Spirulina laxissima, Spirulina curta, and Spirulina spirulinoides.

In step (b), the ethanol was mixed with the Spirulina maxima powder at the ratio of 1:8 to 1:10 (w/w).

The ultrasonic frequency in the ultrasonic pretreatment in step (b) was 30 to 50 kHz.

The ultrasonic pretreatment in step (b) may be performed for 6 to 10 hours.

The extraction in step (c) may be performed for 2 to 6 hours.

The vacuum-concentration in step (d) may be performed at a pressure of 50 to 150 hPa at 30 to 60° C.

The freeze-drying in step (e) may be performed at a pressure of 0 to 10 mTorr at −70 to −40° C.

Further, the present invention provides a pharmaceutical composition for prevention or treatment of degenerative cranial nerve diseases containing a Spirulina extract as an active ingredient which is obtained by preparing a Spirulina powder, adding 50 to 80% (v/v) of ethanol to the Spirulina powder and performing an ultrasonic pretreatment at 15° C. to 35° C., extracting a Spirulina extract at 50° C. to 80° C., and vacuum-concentrating and freeze-drying the extract.

The Spirulina extract may contain chlorophyll a.

The degenerative cranial nerve diseases may be Alzheimer-type dementia, cerebrovascular dementia, Pick disease, Creutzfeldt-Jakob disease, dementia due to head damage, or Parkinson's disease.

The composition may recover the deterioration of cognitive ability as a symptom of the degenerative cranial nerve diseases.

Further, the present invention provides health functional food for prevention or treatment of degenerative cranial nerve diseases containing a Spirulina extract as an active ingredient which is obtained by preparing a Spirulina powder, adding 50 to 80% (v/v) of ethanol to the Spirulina powder and performing an ultrasonic pretreatment at 15° C. to 35° C., extracting a Spirulina extract at 50° C. to 80° C., and vacuum-concentrating and freeze-drying the extract.

The Spirulina extract may contain chlorophyll a.

The degenerative cranial nerve diseases may be Alzheimer-type dementia, cerebrovascular dementia, Pick disease, Creutzfeldt-Jakob disease, dementia due to head damage, or Parkinson's disease.

The composition may recover the deterioration of cognitive ability as a symptom of the degenerative cranial nerve diseases.

Further, the present invention provides a method for treating degenerative cranial nerve diseases including administering to a subject suffering from degenerative cranial nerve diseases a Spirulina extract which is obtained by preparing a Spirulina maxima powder, adding 50 to 80% (v/v) of ethanol to the Spirulina powder and performing an ultrasonic pretreatment at 15° C. to 35° C., extracting a Spirulina extract at 50° C. to 80° C., and vacuum-concentrating and freeze-drying the extract.

The once dose of the Spirulina extract may be 150 mg/kg to 450 mg/kg.

### Advantageous Effects

According to the present invention, in the method for producing the Spirulina extract, it is possible to preserve the total content of chlorophyll included in the extract while increasing the extraction yield of the Spirulina extract.

Further, according to the present invention, it is possible to prevent and treat degenerative cranial nerve diseases such as Alzheimer by providing a pharmaceutical composition and health functional food containing the extracted Spirulina extract as an active ingredient.

### DESCRIPTION OF DRAWINGS

FIG. 1 is a process flowchart of a method for a Spirulina extract according to an embodiment of the present invention.

FIG. 2 is a graph showing comparing escape latency time of a control group and a Spirulina extract-administered group in a Morris water maze test (*p<005, **p<001, ***p<0001).

FIG. 3 is a graph showing comparing latency time of a control group and a Spirulina extract-administered group in a passive avoidance test (*p<005, **p<001, ***p<0001).

FIG. 4 is a graph showing measuring acetylcholinesterase inhibitory activity of a control group and a Spirulina extract-administered group (*p<005, **p<001, ***p<0001).

FIG. 5 is a graph showing measuring expression activation of a brain derived neurotrophic factor (BDNF) of a control group and a Spirulina extract-administered group (*p<005, **p<001, ***p<0001).

FIG. 6 is a graph showing measuring expression activation of a top transcription factor (p-CBEB) of a control group and a Spirulina extract-administered group (*p<005, **p<001, ***p<0001).

FIG. 7 is a graph showing measuring expression activation of a top transcription factor (p-ERK) of a control group and a Spirulina extract-administered group (*p<005, **p<001, ***p<0001).

FIG. 8 is a photograph showing comparing an antibody fluorescence dyeing result of a control group and a Spirulina extract-administered group by tissue-dissecting the brain hippocampus of degenerative cranial nerve diseases-induced experimental mice.

### BEST MODE FOR THE INVENTION

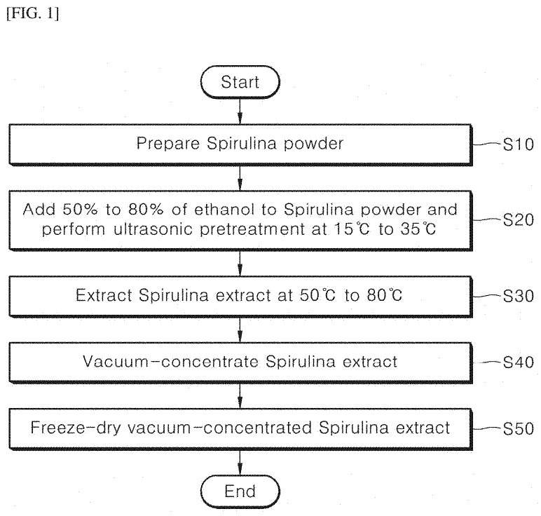

FIG. 1 is a process flowchart of a method for a Spirulina extract according to an embodiment of the present invention.

Referring to FIG. 1, a Spirulina extract according to an embodiment of the present invention is produced by steps of

(a) preparing a Spirulina powder; (b) adding 50% to 80% of ethanol to the Spirulina powder and performing an ultrasonic pretreatment at 15° C. to 35° C.; (c) extracting a Spirulina extract at 50° C. to 80° C.; (d) vacuum-concentrating the Spirulina extract; and (e) freeze-drying the vacuum-concentrated Spirulina extract.

### MODES FOR THE INVENTION

In the following description, only parts required to understand embodiments of the present invention will be described, and it should be noted that the description of other parts will be omitted within a range without departing from the gist of the present invention.

Terms and words used in the present specification and claims should not be interpreted as being limited to typical or dictionary meanings, but should be interpreted as meanings and concepts which comply with the technical spirit of the present invention, based on the principle that the present inventor can appropriately define the concepts of the terms to describe his/her own invention in the best manner. Therefore, the exemplary embodiments described in the present specification and the configurations illustrated in the drawings are merely the most preferred embodiment of the present invention and are not intended to represent all of the technical ideas of the present invention, and thus, it should be understood that various equivalents and modifications capable of replacing the exemplary embodiments at the time of this application.

Method for Producing Spirulina Extract

FIG. 1 is a process flowchart of a method for a Spirulina extract according to an embodiment of the present invention.

Referring to FIG. 1, a Spirulina extract according to an embodiment of the present invention is produced by steps of (a) preparing a Spirulina powder; (b) adding 50% to 80% of ethanol to the Spirulina powder and performing an ultrasonic pretreatment at 15° C. to 35° C.; (c) extracting a Spirulina extract at 50° C. to 80° C.; (d) vacuum-concentrating the Spirulina extract; and (e) freeze-drying the vacuum-concentrated Spirulina extract.

First, the Spirulina powder is prepared (S10).

Spirulina has a spiral shape as blue-green algae and has a size of a width of 10 μm and a length of about 300 to 500 μm to observe each cell with naked eyes. Spirulina has the same etymology as spiral and both words are derived from Latin with the meaning of a twisted or spiral type. Spirulina was known since Spirulina, which grows wild in the Erangarde Lake near the Ethiopia, was announced in the International Conference on Applied Microbiology in 1967. This new plant is similar to Chlorella, while has a very much protein content and a very good digestive absorption rate, is easily cultivated and harvested, and has strong alkaline. The marine algae are classified into blue, green, red and brown algae according to a main pigment, and Spirulina is a kind of blue-green algae, wherein the color is a color exhibited by phycocyanin (blue) of chlorophyll (green) in the cells.

The Spirulina is currently known to have an atopic prevention effect, known to have a skin anti-aging effect, and known to have an effect of improving fat metabolism, but the studies of other physiological activities are still inadequate.

Accordingly, the present inventors have found a therapeutic agent for treating and preventing degenerative cranial nerve diseases with an excellent treatment effect while being stable in the human body and confirmed that a Spirulina extract have these effects, and then completed the present invention.

Particularly, in the present invention, it was confirmed that the Spirulina extract has an excellent effect of improving cognitive ability and high activity of protecting cranial nerve cells to have an excellent effect of treating or preventing degenerative cranial nerve diseases.

The Spirulina used therein may be selected from the group consisting of Spirulina maxima, Spirulina platensis, Spirulina geitleri, Spirulina Siamese, Spirulina major, Spirulina subsalsa, Spirulina princes, Spirulina laxissima, Spirulina curta, Spirulina spirulinoides, and may use preferably Spirulina maxima or Spirulina platensis, more preferably Spirulina maxima.

The Spirulina powder may be a dry-powdered state or a lyophilized state, but is not limited thereto.

50% to 80% of ethanol is added to the Spirulina powder and the ultrasonic pretreatment is performed at 15° C. to 35° C. (S20).

In order to obtain the Spirulina extract, the Spirulina powder is dissolved using ethanol as a solvent. The ethanol may use preferably 50% to 80% of ethanol, more preferably 70% of ethanol.

However, the technical idea of the present invention is not limited thereto, and pharmaceutically acceptable organic solvents may be used. For example, various solvents such as purified water, alcohols having carbon atoms 1 to 4 including methanol, ethanol, propanol, isopropanol, butanol, etc., acetone, ether, benzene, chloroform, ethyl acetate, methylene chloride, hexane, and cyclohexane, and the like may be used alone or in combination.

The Spirulina powder and the ethanol may be mixed at a ratio of 1:8 to 1:10 (w/w).

The ultrasonic pretreatment is performed in the solution dissolved with the Spirulina powder. When extracting after the ultrasonic pretreatment, it is preferred that a total chlorophyll content contained in the Spirulina extract is higher than that of the non-pretreatment and the Spirulina extract from which impurities are removed may be obtained.

The ultrasonic pretreatment may be performed at a frequency of 30 to 50 kHz, preferably 40 kHz.

The ultrasonic pretreatment may be performed at a temperature of 15° C. to 35° C., preferably room temperature (25° C.).

Further, the ultrasonic pretreatment may be performed for 6 to 10 hours, preferably 8 hours.

When the pretreatment condition is less than the range, the effect by the ultrasonic pretreatment is slight, and when the pretreatment condition is more than the range, it is not preferred in that a Spirulina extract modified to a structure which is not effective on improvement of cognitive ability may be obtained.

The Spirulina extract is extracted at 50° C. to 80° C. (S30).

The extraction may be performed at 50° C. to 80° C., but preferably performed at 65° C.

As such, in the method for producing the Spirulina extract according to the present invention, the ultrasonic pretreatment is performed before extraction, and the temperatures at which the ultrasonic pretreatment and the extraction are performed are differently set, thereby increasing the extraction yield and increasing the total content of chlorophyll contained in the extract.

Next, the Spirulina extract is vacuum-concentrated (S40).

The vacuum-concentration is preferably performed using a rotary vacuum evaporator, but is not limited thereto.

The vacuum-concentration may be performed at 30° C. to 60° C., preferably 45° C.

The vacuum-concentration may be performed at a pressure of 50 hPa to 150 hPa, preferably 100 hPa.

Finally, the Spirulina extract is freeze-dried (S50).

The freeze-drying may be performed at −70° C. to −40° C., preferably −55° C.

The freeze-drying may be performed at a pressure of 0 mTorr to 10 mTorr, preferably 5 mTorr.

Pharmaceutical Compositions Containing Spirulina Extract as Active Ingredient

The present invention provides a pharmaceutical composition for prevention or treatment of degenerative cranial nerve diseases containing the Spirulina extract produced according to the producing method as an active ingredient.

The Spirulina extract may include chlorophyll a. The chlorophyll a is a kind of anabolic pigments of organisms using photosynthesis, exists in the chlorophyll in a cell, and exists to be bound with protein or lipoprotein in a natural state.

The chlorophyll a contains polyphenol, and the polyphenol is characterized by having two or more phenol groups in a molecule as a kind of chemical materials found in plants. The polyphenol has an antioxidant effect of changing active oxygen (hazardous oxygen) in the human body to a harmless material to exhibit an excellent effect of preventing degenerative cranial nerve diseases such as Alzheimer-type dementia, cerebrovascular dementia, Pick disease, Creutzfeldt-Jakob disease, dementia due to head damage, or Parkinson's disease.

A formulation form of the pharmaceutical composition may be granules, powders, tablets, coating tablets, capsules, suppositories, solutions, syrups, juice, suspensions, emulsions, drops, injectable liquids, or the like. For example, for formulation in the form of tablets or capsules, the active ingredient may be combined with an oral non-toxic pharmaceutically acceptable inert carrier such as ethanol, glycerol, water, and the like.

Further, if desired or necessary, suitable binders, lubricants, disintegrants and coloring agents may also be included as a mixture. The suitable binder includes natural sugar such as starch, gelatin, glucose or beta-lactose, natural and synthetic gums such as corn sweetener, acacia, tragacanth or sodium oleate, sodium stearate, magnesium stearate, sodium benzoate, sodium acetate, sodium chloride, and the like, but is not limited thereto.

The disintegrant is not limited thereto, but includes starch, methylcellulose, agar, bentonite, xanthan gum, or the like. In the composition formulated with a liquid solution, the pharmaceutically acceptable carrier is suitable for sterilization and living bodies and may use saline, sterilized water, ringer's solution, buffered saline, albumin injection solution, dextrose solution, maltodextrin solution, glycerol, ethanol and at least one of these ingredients in combination, and if necessary, may add other general additives such as antioxidants, buffers, bacteriostatic agents, or the like.

In addition, the composition may be prepared in injectable formulations such as aqueous solutions, suspensions, and emulsions, pills, capsules, granules, or tablets by further adding a diluent, a dispersant, a surfactant, a binder, and a lubricant. Furthermore, as a suitable method of this field, the composition may be prepared preferably according to each disease or ingredient using a method disclosed in Remington's Pharmaceutical Science, Mack Publishing Company, Easton Pa.

The pharmaceutical composition according to the present invention may treat or prevent degenerative cranial nerve diseases through the improvement of the cognitive ability.

The degenerative cranial nerve diseases may be Alzheimer-type dementia, cerebrovascular dementia, Pick disease, Creutzfeldt-Jakob disease, dementia due to head damage, or Parkinson's disease.

Health Functional Food Containing Spirulina Extract as Active Ingredient

The present invention provides health functional food for prevention or treatment of degenerative cranial nerve diseases containing the Spirulina extract produced according to the producing method as an active ingredient.

The Spirulina extract may include chlorophyll a. The chlorophyll a is a kind of anabolic pigments of organisms using photosynthesis, exists in the chlorophyll in a cell, and exists to be bound with protein or lipoprotein in a natural state.

The chlorophyll a contains polyphenol, and the polyphenol is characterized by having two or more phenol groups in a molecule as a kind of chemical materials found in plants. The polyphenol has an antioxidant effect of changing active oxygen (hazardous oxygen) in the human body to a harmless material to exhibit an excellent effect of preventing degenerative cranial nerve diseases such as Alzheimer-type dementia, cerebrovascular dementia, Pick disease, Creutzfeldt-Jakob disease, dementia due to head damage, or Parkinson's disease.

The health functional food may be produced and processed in the form of tablets, capsules, powders, granules, liquids, and pills.

In the present invention, the “health functional food” refers to food produced and processed using raw materials or ingredients with functionality, which are useful for the human body according to the Art on Health Functional Foods No. 6727, and means food taken for adjusting nutrients for the structures and functions of the human body or obtaining an useful effect on health applications such as physiological actions.

The health functional food of the present invention may include conventional food additives, and whether it is suitable as a food additive, unless otherwise noted, is determined by scales and criteria of the corresponding items according to the general provisions, general testing methods, and the like of the Korean Food Additives Codex approved by the Korea Food & Drug Administration.

The items disclosed in the “Korean Food Additives Codex” may include, for example, chemical composites such as ketones, glycine, calcium citrate, nicotinic acid, cinnamic acid, and the like; natural additives such as desensitizing dye, licorice extract, crystal cellulose, Kaoliang color, guar gum, and the like; mixed formulations such as sodium L-glutamic acid formulations, alkali agents for noodles, preservative formulations, tar color formulations, etc.

For example, the health functional food in the form of tablets may produced by granulating a mixture of mixing the Spirulina extract as the active ingredient with an excipient, a binder, a disintegrant, and other additives, and then compression-molding the mixture with a slip modifier and the like or directly. In addition, the health functional food in the form of tablets may contain a flavors enhancer or the like as needed.

In the health functional food in the form of capsules, a hard capsule agent may be produced by filling a mixture of mixing the Spirulina extract as the active ingredient of the present invention with an additive such as an excipient or the like in a general hard capsules, and a soft capsule agent may be produced by filling a mixture of mixing the Spirulina extract with an additive such as an excipient or the like in a capsule material such as gelatin. The soft capsule agent may contain a plasticizer such as glycerin or sorbitol, a colorant, a preservative, and the like if necessary.

The health functional food in the form of pills may be produced by molding a mixture of mixing the Spirulina extract as the active ingredient of the present invention with an excipient, a binder, a disintegrant, and the like by existing known methods, and may be coated with white sugar or other coating agents or surface-coated with a material such as starch and talc, if necessary.

The health functional food in the form of granules may be produced by granulizing a mixture of mixing the Spirulina extract as the active ingredient of the present invention with an excipient, a binder, a disintegrant, and the like by existing known methods and may contain a flavoring agent, a flavors enhancer, and the like if necessary.

The health functional food may be beverages, meat, chocolate, foods, confectionery, pizza, ramen, other noodles, gums, candies, ice creams, alcohol beverages, vitamin composites, health food supplements, and the like.

The health functional food according to the present invention may treat or prevent degenerative cranial nerve diseases through the improvement of the cognitive ability.

The degenerative cranial nerve diseases may be Alzheimer-type dementia, cerebrovascular dementia, Pick disease, Creutzfeldt-Jakob disease, dementia due to head damage, or Parkinson's disease.

Method for Treating Degenerative Cranial Nerve Diseases

The present invention provides a method for treating degenerative cranial nerve diseases including administering a Spirulina extract in a pharmaceutically effective amount to a subject.

The degenerative cranial nerve diseases may be dementia, Alzheimer-type dementia, cerebrovascular dementia, Pick disease, Creutzfeldt-Jakob disease, dementia due to head damage, or Parkinson's disease.

The subject may include all animals, including humans.

The Spirulina extract may further contain one or more active ingredients exhibiting the same or similar functions.

The administration can be performed by oral administration, or parenteral administration such as subcutaneous injection, intravenous injection, or intramuscular injection, and may be used in the form of general medical preparations.

A dosage unit of the administration may contain 1, 2, 3 or 4 times of an individual dose, or may contain ½, ⅓ or ¼ times thereof. The individual dose preferably contains an amount in which an effective drug is administered once, which usually corresponds to all, ½, ⅓ or ¼ times of a general daily dose.

The dose of administration may vary depending on the age, weight, and gender of a patient, a dosage form, a health condition and a disease degree, and may be divided and administered once to several times a day at a certain time interval depending on the judgment of doctors or pharmacists.

A single dose may be 150 mg/kg to 450 mg/kg. When the dose is less than the dose range, no significant effect can be obtained, and when the dose is more than the dose range, the dose is not only non-economical, but also is out of a common dose range, so that undesirable side effects may occur, and thus it is preferable to be set in the above range.

The frequency of administration is not particularly limited, but the composition may be administered once a day or several times a day by dividing the dose.

### EXAMPLES

Hereinafter, the present invention will be described in detail with reference to Examples for specific description. However, Examples according to the present invention may be modified in various forms, and it is not interpreted that the scope of the present invention is limited to the following Examples. Examples of the present invention will be provided for more completely explaining the present invention to those skilled in the art.

### Example 1: Production of Spirulina Extract

To obtain a Spirulina extract, 100 g of Spirulina maxima in the form of dried powder was mixed with 70% ethanol at a ratio of 1:10 (w/w). Thereafter, at a frequency of 40 kHz, ultrasonic pretreatment was performed at 25° C. for 8 hours and then the Spirulina extract was extracted at 65° C. for 4 hours. The Spirulina extract obtained after extraction was vacuum-concentrated at a pressure of 100 hPa at 45° C. and then freeze-dried at a pressure of 5 mTorr at −55° C. to be prepared in a powder form and then the following experiment was performed.

### Experiment Example 1: Comparison of Yield of Spirulina Extract Extracting Process

By the Spirulina extract extracting process of Example 1, a conventional heat-water extracting process, and a 70% and 100% ethanol extracting processes, extraction yields and a total chlorophyll contents were compared (Table 1).

TABLE 1

| | Extraction | Total chlorophyll |
|---|---|---|
| Extraction condition | yield (%) | content (mg/g) |
| Example 1 | 11.6 | 17.5 |
| Comparative Example 1 | 13.2 | 3.51 |
| (Water, 100° C., 24 h) | | |
| Comparative Example 2 | 12.4 | 7.86 |
| (70% EtOH, 80° C., 24 h) | | |
| Comparative Example 3 | 11.1 | 13.42 |
| (100% EtOH, 80° C., 24 h) | | |

It was confirmed that the yields of the extraction processes of Example 1 and Comparative Examples 1 to 3 were distributed between 10% to 13%.

The extraction yield of the Spirulina extract obtained by Example 1 was approximately 11.6%, which was similar to or higher than that of the existing other processes. In addition, in the case of the extracting processes of Comparative Example 1 and Comparative Example 2, which had a higher yield than 11.6%, it was confirmed that the total chlorophyll content as an indicator component was lower than that of Example 1.

Therefore, it was confirmed that while the extracting process according to Example 1 was similar to or higher than that of the existing other processes, the total content of chlorophyll contained in the extract may be increased.

### Experiment Example 2: Measurement of Improvement Ability of Cognitive Disorders Caused by Aβ1-42 of Spirulina Extract

Experimental Animals and Drug Administration

The deterioration of cognitive ability of Aβ1-42 (Amyloid Beta 1-42) was evaluated in 4-week-old male mice. ICR mice (Bio Link, Eumseong-gun, Chungbuk, Korea) were purchased, adapted to an experimental animal room for one week, and randomly consisted of 6 mice in each group. As experimental groups, a group administered with the Spirulina extract (hereinafter, SM70EE) produced by Example 1 by concentration and a group administered with chlorophyll a were used. As a control group, a group without treating a Spirulina extract, and as a comparative group, a group administered with scopolamine causing dementia and a group administered with donepezil as a dementia therapeutic agent were used. During the adaptation period, the feed and water were freely supplied without limitation, and the temperature of 22±2° C. and the humidity of 50±10% were maintained, and the contrast was adjusted to a period of 12 hours (09:00 to 21:00).

Method of Evaluating Cognitive Ability

(1) Morris Water Maze Test

In Experimental Example above, a passive avoidance analysis evaluation test was conducted by applying a Morris's method. Milk-mixed water (water temperature: 20±1° C.) was filled in a water maze pool (diameter: 90 cm, height: 45 cm). The pool was divided into four equal parts, and a white platform (diameter: 6 cm, height: 29 cm) was disposed at a center of one part, and the platform was drowned by about 1 cm and not visible on the surface of the water. For adaptation, only the swimming practice was performed for 60 seconds without a platform on the first day of the experiment, a test trial was performed for 4 days in the pool disposed with the platform from the second day, and then escape latency time which was time taken for experimental mice administered with a drug to escape on the platform was measured.

Experimental mice that did not escape from the platform within 120 seconds were placed on the platform directly by a tester, and maintained in an escape condition for 10 seconds. The position of the platform was fixed for four days and the positions of entering water of the experimental mice were different.

Sample administration was performed daily for four days from the second day, scopolamine (1 mg/kg) was dissolved in physiological saline and prepared, and the prepared scopolamine was subcutaneously injected before 30 minutes of training to cause dementia. Before 1 hour of scopolamine administration, a Spirulina Extract (SM70EE) (SM70EE) (200, 400 mg/kg), chlorophyll a (10 mg/kg), and donepezil (1 mg/kg) as a positive control group were orally administered, and what the sample affected the dementia induced by scopolamine was confirmed.

(2) Passive Avoidance Test

An avoidance box (40×20×20 cm) of a passive avoidance measuring device was divided into a light box and a dark box, and a door was installed between the rooms so that the experimental mice moved. When mice in a light box, in which a stainless steel bar of a 3 mm thickness was installed on the bottom of the box at an interval of 0.5 cm, entered a dark box, the electric stimulation of 0.1 mA/10 g body weight was performed through the stainless steel bar. After 24 hours, the same test was performed to measure the time while the experimental mice stayed in a bright room as an index of remembering the training of the previous day. The sample was administered orally, and scopolamine was subcutaneously administered.

Before 120 minutes of experiment, a Spirulina Extract (SM70EE) (SM70EE) (200, 400 mg/kg), chlorophyll a (10 mg/kg), and donepezil (1 mg/kg) as a positive control group were orally administered, and after 90 minutes, in a control group, normal saline was subcutaneously administered and in another group, scopolamine (1 mg/kg) was subcutaneously administered. The scopolamine was administered and after 30 minutes, the test was conducted. After 24 hours, the same test was conducted, and the escape latency time while the experimental mice moved was measured. If there was no movement of the experimental mice for 180 seconds, the test was stopped.

Experiment Results

(1) Escape Latency Time for 4 Days in Morris Water Maze Test

In a Morris water maze test for 4 days, spatial cognitive ability improvement activity of the Spirulina extract (SM70EE) was confirmed.

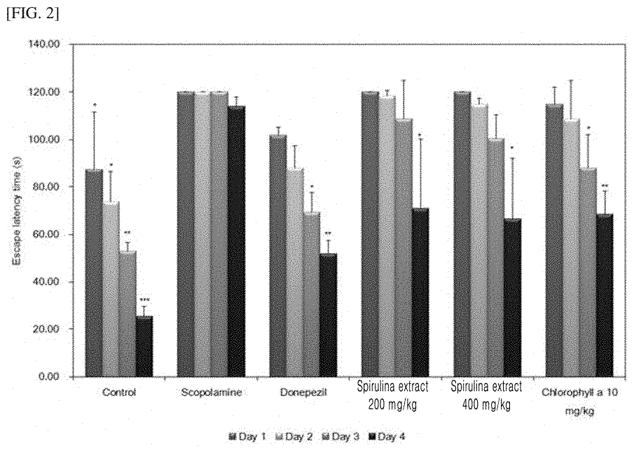

FIG. 2 is a graph showing comparing escape latency time of a control group and a Spirulina extract-administered group in a Morris water maze test (*p<005, **p<001, ***p<0001).

Referring to FIG. 2, in the case of the control group, the escape latency time was significantly reduced from day 1 to day 4, and in the group treated with only scopolamine, the escape latency time was increased. The Spirulina extract (SM70EE) decreased the escape latency time increased by the scopolamine in a concentration dependent manner at day 4, and at both concentrations of 200 and 400 mg/kg, low escape latency time at a similar level was shown, but at the concentration of 400 mg/kg, it was confirmed that the escape latency time of 66.39±25.70 seconds was lowest. In the case of chlorophyll a (10 mg/kg), it was also confirmed that the escape latency time was decreased in a concentration dependent manner.

(2) Latency Time in Passive Avoidance Test

The Memory improvement activity of the Spirulina extract (SM70EE) was measured through a passive avoidance test.

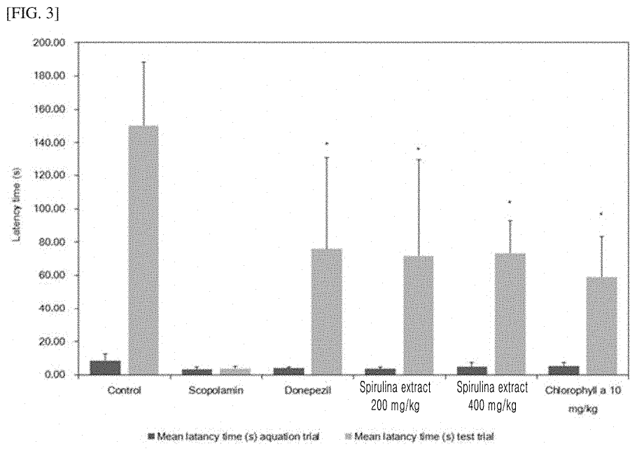

FIG. 3 is a graph showing comparing latency time of a control group and a Spirulina extract-administered group in a passive avoidance test (*p<005, **p<001, ***p<0001).

Referring to FIG. 3, in the case of experimental mice treated with scopolamine, the latency time was significantly reduced compared to the control group. The Spirulina extract (SM70EE) significantly increased the latency time reduced by scopolamine at concentrations of 200 and 400 mg/kg, and at the concentration of 400 mg/kg, it was confirmed that the latency time reduced to 73.33±19.35 seconds was the highest. In addition, chlorophyll a (10 mg/kg) also increased the latency time reduced by scopolamine.

### Experiment Example 3: Inhibitory Activity Measurement of Acetylcholinesterase (ACNE) of Spirulina Extract

In this Experimental Example, the hippocampus of the mice of each experimental group used in an animal experiment of Experimental Example 2 was extracted to measure acetylcholinesterase inhibitory activity.

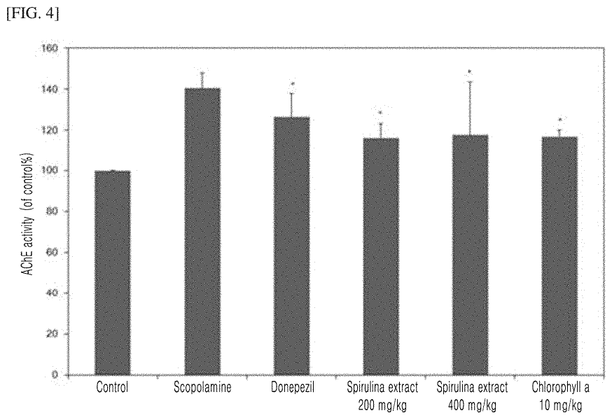

FIG. 4 is a graph showing comparing acetylcholinesterase inhibitory activity of a control group and a Spirulina extract-administered group (*p<005, **p<001, ***p<0001).

Acetylcholinesterase, as an enzyme that decomposed acetylcholine, a neurotransmitter in a brain's cholinergic system, reduced memory and cognitive ability when the activity increased.

A phosphate buffer was added in the hippocampus of each control and experimental group mice, homogenized through a homogenizer, and centrifuged at a 12,000 rpm condition for 20 min at 4° C. and then a supernatant was taken. In a 96 well plate, the supernatant, a standard, and a blank were divided by 50 μL. 20 μM of an acetylcholinesterase assay solution was added by 50 μL, and then cultured for 15 min, and the absorbance was measured at 410 nm.

Referring to FIG. 4, in the case of experimental mice administered with scopolamine, the acetylcholinesterase activity of 140.39±7.55% was measured and increased compared to the control group. In the case of the Spirulina extract (SM70EE), the acetylcholinesterase activities of 116.16±7.11% and 117.55±25.96% were measured for each concentration of 200 and 400 mg/kg, respectively, and the acetylcholinesterase activity increased by scopolamine was significantly reduced. As a result, it was confirmed that the Spirulina extract (SM70EE) inhibited the activity of acetylcholinesterase to affect the improvement of cognitive ability.

In addition, as a result of confirming an optimal dose, at the concentrations of 200 mg/kg and 400 mg/kg, the cognitive ability activity was increased in a concentration dependent manner. However, since its deviation is slight, it is evaluated that the activity is similar at two concentrations, so that it is determined that the dose of 200 mg/kg is suitable for the activity improvement of cognitive ability in consideration of the economics in production.

### Experiment Example 4: Measurement of Expression of Neurotrophic Factor (BDNF) and Top Transcription Factors (CREB and ERK) of Spirulina Extract

In this Experimental Example, the hippocampus of mice of each experimental group used in the animal experiment of Experimental Example 2 was extracted to measure the expression of a neurotrophic factor (BDNF) and the expression of top transcription factors (CREB and ERK).

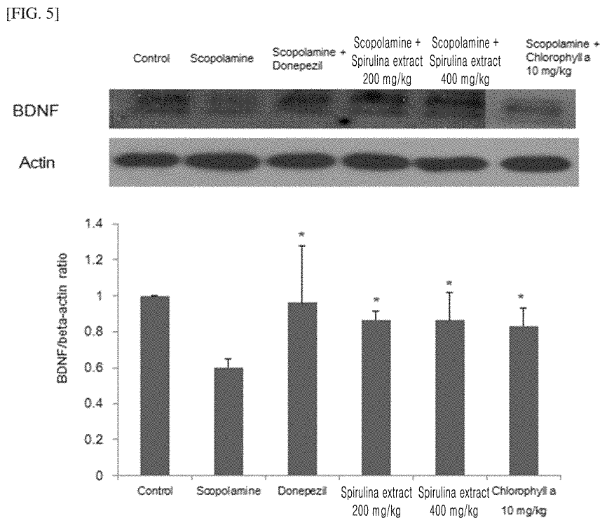

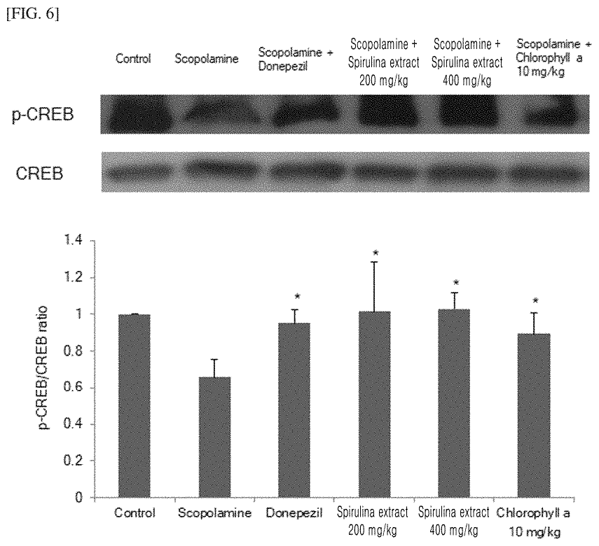

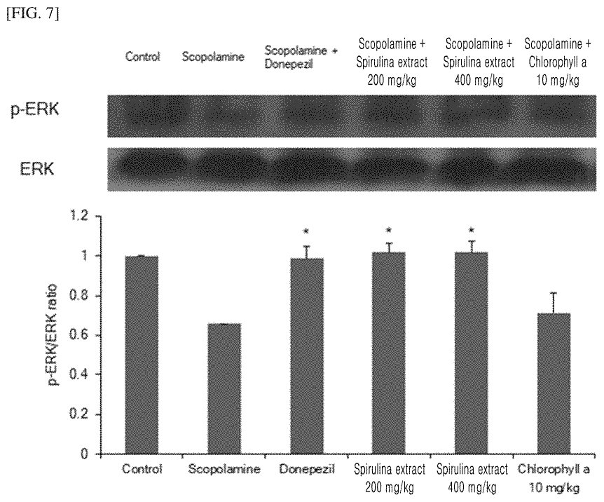

FIG. 5 is a graph showing measuring expression activation of a brain derived neurotrophic factor (BDNF) of a control group and a Spirulina extract-administered group (*p<005, **p<001, ***p<0001), FIG. 6 is a graph showing measuring expression activation of a top transcription factor (p-CBEB) of a control group and a Spirulina extract-administered group (*p<005, **p<001, ***p<0001), and FIG. 7 is a graph showing measuring expression activation of a top transcription factor (p-ERK) of a control group and a Spirulina extract-administered group (*p<005, **p<001, ***p<0001).

The BDNF generates new neurites to reinforce the connections between neurons and increase the number of connections, thereby increasing the cognitive ability, and the CREB acts as a top transcription factor of the BDNF expression as a gene required during learning and memory processes.

A phosphate buffer was added in the hippocampus of mice in control and experimental groups, homogenized through a homogenizer, and centrifuged at a 12,000 rpm condition for 20 min at 4° C. and then a supernatant was taken. In a 96 well plate, the supernatant, a standard, and a blank were dispensed by 100 μL, and cultured in an incubator at 37° C. for 1 hr 30 min. The supernatants in the plate were removed and 100 μL of a biotinylated anti-Mouse BDNF antibody working solution was added and cultured in an incubator at 37° C. for 1 hr. The supernatants in the plate were removed and washed three times with 0.01 M of a phosphate buffer. 100 μL of an ABC working solution was added and reacted in the incubator at 37° C. for 30 min. After washing 5 times with 0.01 M of the phosphate buffer, 90 μL of a TMB color development agent was added and reacted at 37° C. for 20 to 25 min and the measured with then absorbance of 450 nm.

Referring to FIGS. 5 to 7, in the case of experimental mice administered with scopolamine, an expression value lower than the BDNF expression value in the control group was shown. It was confirmed that the amount of BDNF, which was a neurotrophic factor reduced by scopolamine, was significantly activated in the hippocampus of the experimental mice administered with the Spirulina extract (SM70EE) at 200 and 400 mg/kg. In addition, it was confirmed that the Spirulina extract (SM70EE) significantly activated the amount of p-CREB and the result of inducing the ERK activity was confirmed.

Therefore, it can be confirmed that the Spirulina extract (SM70EE) increases the expression levels of the reduced neurotrophic factor (BDNF) and the top transcription factors (CREB and ERK) to affect the improvement of cognitive ability.

In addition, as a result of confirming an optimal dose through an animal behavior test, at the concentrations of 200 mg/kg and 400 mg/kg, the cognitive ability activity was increased in a concentration dependent manner. However, since its deviation is slight, it is evaluated that the activity is similar at two concentrations, so that it is determined that the dose of 200 mg/kg is suitable for the activity improvement of cognitive ability in consideration of the economics in production.

### Experiment Example 5: Measurement of Inhibitory Activity of Acetylcholinesterase (ACNE), Expression of Top Transcription Factor (CREB), and Expression Ability of Glial Fibrillary Acidic Protein (GFAP) of Spirulina Extract Using Antibody Fluorescent Staining Method

In Experimental Example, in order to confirm the inhibitory activity ability of acetylcholinesterase (AChE), the expression ability of a top transcription factor (CREB), and the expression ability of a glial fibrillary acidic protein (GFAP) in the brain hippocampus of the Spirulina extract, the brain hippocampus of the experimental mice was tissue-dissected and strained through antibody fluorescent staining.

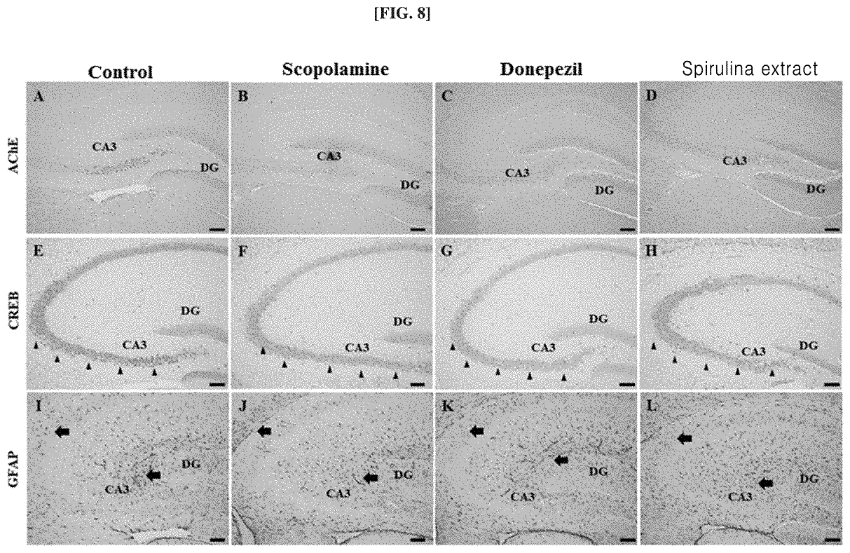

FIG. 8 is a photograph showing comparing an antibody fluorescence dyeing result of a control group and a Spirulina extract-administered group by tissue-dissecting the brain hippocampus of degenerative cranial nerve diseases-induced experimental mice.

Referring to FIG. 8, in the group administered with scopolamine, it was observed that the activity of acetylcholinesterase was increased through the entire hippocampus and the cerebral cortex. In the group administered with donepezil and the group administered with the Spirulina Extract, it was confirmed that the activity of acetylcholinesterase was deteriorated as compared with the group administered with scopolamine.

Further, it was confirmed that in the group administered with scopolamine, the activity of CREB was significantly reduced compared with the control group, but in the groups administered with donepezil and the Spirulina extract, the reduction degree of CREB activity was slight.

Meanwhile, the glial fibrillary acidic protein (GFAP) is presented in astrocytes, and used as a marker of these astrocytes. To identify the expression degree of astrocytes, the immunostaining of GFAP was performed.

Referring to FIG. 8, in the control group, it was confirmed that GFAP-positive astrocytes were distributed evenly in the hippocampus of the brain, and some astrocytes were in a full form, that is, activated state in the dentate gyrus (DG). In the entire hippocampus of the scopolamine-administered group, the positive response of GFAP was increased, while in the groups administered with donepezil and the Spirulina extract, it was observed that the activated astrocytes were reduced in the dentate gyrus (DG) and the hippocampus portion was stable.

These results are judged as a result that the Spirulina extract reduces the body waste of causing degenerative brain diseases in and out of the brain cells.

Hereinabove, specific embodiments for the method for producing the Spirulina extract according to an embodiment of the present invention, the pharmaceutical composition and the health functional food for prevention or treatment of degenerative cranial nerve diseases containing the Spirulina extract, and the method for treating the degenerative cranial nerve diseases using the Spirulina extract have been described, but it will be apparent that many modification can be made within the limit without departing from the scope of the present invention.

Therefore, the scope of the present invention should not be limited to the embodiments and should be defined by the appended claims and equivalents to the appended claims.

In other words, the embodiments described above are illustrative in all aspects and should be understood as not being restrictive, and the scope of the present invention is represented by appended claims to be described below rather than the detailed description, and it is to be interpreted that the meaning and scope of the appended claims and all changed or modified forms derived from the equivalents thereof are included within the scope of the present invention.

1. A method for producing a Spirulina extract comprising:
(a) preparing a Spirulina maxima powder;
(b) adding 50 to 80% (v/v) of ethanol to the Spirulina maxima powder and performing an ultrasonic pretreatment at 15° C. to 35° C.;
(c) extracting a Spirulina maxima extract at 50° C. to 80° C.;
(d) vacuum-concentrating the Spirulina maxima extract; and
(e) freeze-drying the vacuum-concentrated Spirulina maxima extract.

2. A method for producing a Spirulina extract comprising:
(a) preparing a Spirulina maxima powder;
(b) mixing 50 to 80% (v/v) of ethanol with the Spirulina maxima powder at a ratio of 1:8 to 1:10 (w/w) and performing an ultrasonic pretreatment at 15° C. to 35° C. for 6 to 10 hours;
(c) extracting a Spirulina maxima extract at 50° C. to 80° C. for 2 to 6 hours;
(d) vacuum-concentrating the Spirulina maxima extract; and
(e) freeze-drying the vacuum-concentrated Spirulina maxima extract.

3. The method for producing the Spirulina extract of claim 1, wherein in step (b), the ethanol is mixed with the Spirulina maxima powder at the ratio of 1:8 to 1:10 (w/w).

4. The method for producing the Spirulina extract of claim 1, wherein the ultrasonic frequency in step (b) is 30 to 50 kHz.

5. The method for producing the Spirulina extract of claim 1, wherein the ultrasonic pretreatment in step (b) is performed for 6 to 10 hours.

6. The method for producing the Spirulina extract of claim 1, wherein the extraction of the Spirulina maxima extract in step (c) is performed for 2 to 6 hours.

7. The method for producing the Spirulina extract of claim 1, wherein the vacuum-concentration in step (d) is performed at a pressure of 50 to 150 hPa at 30 to 60° C.

8. The method for producing the Spirulina extract of claim 1, wherein the freeze-drying in step (e) is performed at a pressure of 0 to 10 mTorr at −70 to −40° C.

9. A pharmaceutical composition for improvement of cognitive ability containing a Spirulina extract as an active ingredient which is obtained by preparing a Spirulina maxima powder, adding 50 to 80% (v/v) of ethanol to the Spirulina maxima powder and performing an ultrasonic pretreatment at 15° C. to 35° C., extracting a Spirulina maxima extract at 50° C. to 80° C., and vacuum-concentrating and freeze-drying the extract.

10. The pharmaceutical composition for improvement of cognitive ability of claim 9, wherein the Spirulina maxima powder contains chlorophyll a.

11. The pharmaceutical composition for improvement of cognitive ability of claim 9, wherein the composition recovers the deterioration of cognitive ability as a symptom of the degenerative cranial nerve diseases.

12. Health functional food for improvement of cognitive ability containing a Spirulina extract as an active ingredient which is obtained by preparing a Spirulina maxima powder, adding 50 to 80% (v/v) of ethanol to the Spirulina maxima powder and performing an ultrasonic pretreatment at 15° C. to 35° C., extracting a Spirulina maxima extract at 50° C. to 80° C., and vacuum-concentrating and freeze-drying the extract.

13. The health functional food for improvement of cognitive ability of claim 12, wherein the Spirulina maxima powder contains chlorophyll a.

14. The health functional food for improvement of cognitive ability of claim 12, wherein the health functional food recovers the deterioration of cognitive ability as a symptom of the cranial nerve diseases.
